(19) United States
(12) Patent Application Publication
(10) Pub. No.: US 2026/0131188 A1
(43) Pub. Date: May 14, 2026

(54) Exercise Apparatus Including Stimulation and Methods of Use

(71) Applicant: TS Medical, LLC, McLean, VA (US)

(72) Inventors: Mary Anne Tarkington, McLean, VA (US); David G. Matsuura, Solana Beach, CA (US); Jacob A. Moebius, San Diego, CA (US)

(73) Assignee: TS Medical, LLC, McLean, VA (US)

(21) Appl. No.: 18/877,211

(22) PCT Filed: Jun. 30, 2023
(86) PCT No.: PCT/US2023/069520

§ 371 Date: Dec. 19, 2024

Related U.S. Application Data
(60) Provisional application No. 63/357,162, filed on Jun. 30, 2022.
(60) Provisional application No. 63/357,345, filed on Jun. 30, 2022.

(51) Int. Cl. A63B 21/00; A61H 23/00; A61N 1/36; A63B 23/08

(52) U.S. Cl. CPC A63B 21/4034; A61H 23/00; A63B 21/4047; A63B 23/08; A61H 2201/10; A61H 2205/12; A61N 1/36034; A63B 2213/004; A63B 2220/803; A63B 2225/64

(57) ABSTRACT

An exercise and stimulation device may include an exercise apparatus including a resistive member resisting a motion of a part of a user's body; and a stimulation device removably engaging both the resistive member and the part of the user's body for providing at least two different stimulations to the user's body, where the at least two different stimulations are different than one another.

### CROSS REFERENCE TO RELATED APPLICATIONS

This application claims benefit of U.S. Provisional Application No. 63/357,345, filed Jun. 30, 2022, entitled EXERCISE APPARATUS INCLUDING STIMULATION, and U.S. Provisional Application No. 63/357,162, filed Jun. 30, 2022, entitled EXERCISE APPARATUS INCLUDING STIMULATION, both of which are hereby incorporated by reference in their entireties.

### BACKGROUND

Global industrialization and recent dominance of the digital sphere have profoundly impacted all aspects of our lives, from transportation and the workplace, to our leisure time. These shifts in lifestyle have been marked by sedentarism, obesity, and an emerging global public health crisis in a rapidly aging population. Reversing this trend requires a multifaceted approach, a key component of which will be exercise, best provided through simple, focused programs.

Cardiovascular disease (CVD) remains the leading cause of disease burden in the world, with cases nearly doubling in the past 30 years to 523 million in 2019. The CVD burden continues its decades-long rise for almost all countries outside high-income countries, and alarmingly, the age-standardized rate of CVD has begun to rise in some high-income countries where it was previously declining.

A common comorbidity of CVD can include compromised circulation, often associated with aging and sedentarism. It can have a broad range of serious adverse health effects that significantly impact both individuals and the healthcare system. Poor circulation can stem from reduced functioning of the arteries, veins, and/or peripheral nerves. Regardless of the pathology, there are certain common health impacts, which can be serious. These may range from superficial thrombophlebitis and edema to more serious issues like cognitive impairment, non-healing ulcers, and chronic pain. In cases of severe venous insufficiency, pooling of blood in the veins can lead to deep vein thrombosis, lymphedema, and more.

In some cases, aging and disease can compromise circulation by directly impacting vascular endothelial cells. In its normal, healthy state, the blood vessel wall maintains its integrity, inhibits inflammation, and responds to changing hemodynamics. Endothelial cells form the lining of the blood vessels and play a critical role in circulation, regulating vascular tone, cellular adhesion, thromboresistance, smooth muscle cell proliferation and vessel wall inflammation. Mechanoreceptors within endothelial cells are highly sensitive to changes in compression and velocity of blood flow, also known as hemodynamic shear stress, and modify vessel dilation accordingly.

As shear stress changes, a complex biochemical cascade initiates, triggering the production of nitric oxide (NO), prostacyclin, and other molecules, all of which cause vascular smooth muscle relaxation. Prostacyclin also mitigates platelet aggregation. In addition, NO decreases arteriole resistance, which increases capillary flow and blocks pain receptors. It helps to maintain the vascular wall in a quiescent state by inhibiting inflammation, cellular proliferation, and thrombosis. Experts believe that shear stress is the primary factor that maintains this healthy, quiescent, NO-dominated endothelial state.

Aging is correlated with a decrease in the production and bioavailability of NO as a result of endothelial oxidative stress and inflammation. This is part of a biochemical cascade that is generally associated with vascular endothelial dysfunction. Specifically, the production of certain intracellular enzymes (e.g., NADPH and eNOS) increases while the body's natural antioxidant response does not increase in appropriate countermeasure. Essentially, this is a change in the redox dynamics of the endothelium, which activates production of pro-inflammatory cytokines, further suppressing endothelial function. A vicious cycle then takes root: redox changes feed inflammation, which feeds further oxidative stress, etc. At the same time, the cells of the vascular endothelium begin to exhibit genomic instability with age, as DNA damage and telomere dysfunction occur with increasing frequency. Overall, these processes can lead to loss of endothelial dependent dilation, which is predictive of hypertension and other forms of CVD.

Like aging, other risk factors, such as hypertension, diabetes, obesity, hypercholesterolemia, and other inflammatory conditions, trigger the expression of chemokines, cytokines, and adhesion molecules designed to interact with leukocytes and platelets and target inflammation to specific tissues. Prolonged and/or recurrent cardiovascular risk factors can eventually exhaust the protective effect of endogenous anti-inflammatory systems within the endothelium, causing the transition from a quiescent phenotype to one involving the host defense response, ultimately resulting in endothelium dysfunction and loss of integrity.

Like aging and certain health conditions, reduction of physical activity can adversely affect the vascular endothelium. It too generates oxidative stress that feeds into a cycle with inflammation as described above. In short, sedentarism initiates a decrease in blood circulation that leads to a subsequent decline in local shear stress, inhibiting production of NO and vasodilation, leading to a measurable vascular impairment and notable increase in vascular wall stiffness.

In tandem with these vascular changes, the structure and oxidative function of muscles can be adversely affected within as little as two weeks of inactivity and sedentarism. The absence of hemodynamic vascular signaling normally associated with exercise triggers altered gene expression in skeletal muscle. Generally referred to as sarcopenia, there is a loss of skeletal muscle mass, strength and quality. Consequently, after even a few weeks of reduced physical activity, beneficial cardiovascular and metabolic adaptations can be lost, often leading to decreased circulation and difficulty resuming exercise. As such, sedentarism can initiate a downward spiral of severe deconditioning, leading to long-lasting functional deficits.

It should be noted that recent public health measures, including quarantine and self-isolation to prevent spread of COVID-19, have brought about a drastic reduction in mobility of the general population that may have serious, detrimental effects. Evidence suggests that the sedentarism associated with these measures has led to de-conditioning that is associated with self-reported decrease in physical and mental health. This reduced muscle strength and trend toward development of sarcopenia is associated with acute respiratory distress and death, which has direct implications for survival of COVID-19 patients.

Ironically, some experts have stated that promotion of sedentary indoor lifestyle during the COVID-19 pandemic is at odds with the most efficient behavioral interventions known to decrease the vulnerability to severe forms of the disease. It has been established in the literature that physical activity can support humoral and cell-mediated immunity, particularly with respect to defense from viral infection. Indeed, exercise has been shown to modulate immune system plasticity and as such, mitigate the cytokine storm of COVID-19 infection and even enhance baseline antibody response to vaccination. Furthermore, following COVID infection, the effects of exercise can counteract symptoms of “long COVID” such as lethargy and increased thrombosis.

In summary, the changes in hemodynamic vascular signaling that come with aging, disease, and sedentarism may adversely affect the health of muscles and adaptability/dilation of vessels. These phenomena in turn can adversely affect metabolic and immune function.

One important example of this is the progression of the metabolic syndromes seen in association with poor circulation, diabetes, and obesity. Sudden cessation of exercise and decline of skeletal muscle have been associated with rapid onset of insulin resistance and poor muscle glucose utilization. Aging can similarly drive the sequelae. In addition to being associated with the metabolic syndrome, sedentarism is also tied to obesity, which can exacerbate diabetes. Obesity can be regarded as generative of metabolic syndrome in and of itself, as it is known to impair metabolism of lipids and glucose while generally elevating chronic systemic inflammation that is especially deleterious to function of muscle and mitochondria. Thus, emerges sarcopenic-obesity among other conditions, with a worsening prognosis.

As mentioned above, immune system dysfunction is also believed to be a possible result of the decline in vascular health associated with aging, disease and sedentarism. Physical inactivity, in particular has been linked with elevated systemic inflammation, impaired natural killer cell cytolytic activity, reduced T-cell proliferation and reduced cytokine production. Presumably, the aspects of aging and disease that have similar cascades might have similar impact on immune health. The research community has taken a new level of interest in the link among aging, reduced physical activity, and alterations in immune function in light of recent public health policy and understanding should grow in the near term.

Beyond the effects on the immune system and metabolism, the literature suggests that compromised circulation may well have causal links to poor cognition, depression, cancer, cardiovascular disease, thromboembolism, and elevated mortality.

Therefore, there is a need in the art for devices and methods to ensure that the above noted degenerative diseases are prevented by means of an easily accessible exercise device and method which can combine guided resistance exercise with stimulation of a targeted area on a user's body.

### SUMMARY

The instant disclosure is generally directed to a novel integration between resistance exercise and stimulation. For example, the plates and frames of the devices disclosed herein guide the path of motion where they interface with the body; provide resistance to the motion of the exercise; and provides a synergistic effect to the user with application of at least one of mechanical, thermal, or electrical stimuli.

In accordance with aspects of the instant disclosure, an exercise apparatus is disclosed. The exercise apparatus includes a resistive member resisting a motion of a part of a user's body; and a stimulation device removably engaging both the resistive member and the part of the user's body for providing at least two different stimulations to the user's body, where the at least two different stimulations are different than one another.

In accordance with aspects of the instant disclosure, one of the at least two different stimulations can be mechanical stimulation in the form of a vibration frequency range of about 30-60 Hz. The mechanical stimulation can have a duration of approximately 5 minutes. The mechanical stimulation can have an amplitude of approximately 2 mm. The stimulation device can include at least one of a brushless motor, brushed motor, stepper motor, cam, linkage, piston, eccentric rotating mass (ERM) motor, linear resonant actuator (LRA), pump, or solenoid, to provide the mechanical stimulation. The mechanical stimulation can be actuated as function of a motion of the resistive member.

In accordance with aspects of the instant disclosure, one of the at least two different stimulations can be electrical stimulation. The electrical stimulation can be provided via contact with an electrode or a wired fabric disposed on the stimulation device. The electrical stimulation can be selected from the group consisting of transcutaneous nerve stimulation (TENS), interferential therapy (IFT), pulsed electromagnetic field (PEMF), and Neuromuscular electrical stimulation (NMES). The electrical stimulation can be supplied with a frequency in a range of about 10 Hz to about 50 Hz. The electrical stimulation can have a duty cycle of about 1:3.5. The electrical stimulation can be provided as function of a motion of the resistive member.

In accordance with aspects of the instant disclosure, at least one of the at least two different stimulations can be thermal stimulation. The stimulation device can include a thermal stimulation device selected from the group consisting of a thermal resistive wire, an ultrasound generator, a shortwave diathermy (SWD), a microwave diathermy (MWD), a thermal pack, and a thermal compress. The thermal stimulation can be provided as function of a motion of the resistive member.

In accordance with aspects of the instant disclosure, the exercise apparatus can further include a sensor configured to detect a predetermined parameter associated with the user's body; and a controller configured to receive detected data from the sensor and control an operation of stimulation according to at least a part of the detected data. The sensor can detect a motion of or a force applied to the resistive member or a predetermined parameter associated with the user. The sensor can be selected from the group consisting of goniometer, SpO2, pulse rate monitor, temperature sensor, accelerometer, gyroscope, magnetometer, pedometer, pressure sensor, bioimpedance, electrocardiography and electrodermal activity (EDA) sensor. The controller can have a user interface for receiving user inputs to control an operation of the stimulation device. The controller can be a remote controller for operating the stimulation device. In some embodiments, the at least two different stimulations are a predefined program that is associated with an exercise and the user.

In accordance with aspects of the instant disclosure, an exercise apparatus is disclosed. The exercise apparatus can include a pivotable foot rest; a resistive member coupled to and resisting a motion of the pivotable foot rest; a stimulus member engaging with the pivotable foot rest, the stimulus member being configured to: provide a mechanical stimulation to a user's body; provide an electrical stimulation to the user's body; and provide a thermal stimulation to the user's body; a sensor configured to detect a predetermined parameter associated with the user's body; and a controller configured to receive detected data from the sensor and control an operation of the stimulus member according to at least a part of the detected data.

In accordance with aspects of the instant disclosure, the pivotable foot rest can include a casing for partially surrounding the user's body; a liner inserted within the casing for receiving the user's body; and at least one cuff for securing the casing to the user's body. The sensor can be disposed in one of the at least one cuff or the liner. The exercise apparatus can further include a second stimulus member disposed within at least one of the at least one cuff or the liner. The stimulus member can be one of removably engaging top surface of the pivotable foot rest or integrated into the pivotable foot rest.

In accordance with aspects of the instant disclosure, a method of use is disclosed. The method includes providing a pivotable pedal having a resistance member for resisting pivoting of the pivotable pedal; pivoting a part of a user's body to pivot the pivotable pedal; and generating at least two different stimulations to the part of the user's body with a stimulation device.

In accordance with aspects of the instant disclosure, the at least two different stimulations can include two of mechanical stimulation, thermal stimulation, and electrical stimulation. The method can include detecting a predetermined parameter of the user with a sensor associated with the stimulation device; and controlling the at least two different stimulations as a function of the detected predetermined parameter.

In accordance with aspects of the instant disclosure, a stimulus member is disclosed. The stimulus member includes a housing configured to removably engage a resistive exercise device; a stimulation device configured to deliver at least one of a mechanical stimulation, electrical stimulation, or thermal stimulation to a user's body; a sensor configured to detect a predetermined parameter associated with the user's body; and a controller configured to receive detected data from the sensor and control an operation of the stimulation device according to at least a part of the detected data.

In accordance with aspects of the instant disclosure, the stimulation device can deliver mechanical stimulation in the form of a vibration frequency range of about 30-60 Hz. The mechanical stimulation can have a duration of approximately 5 minutes. The mechanical stimulation can have an amplitude of approximately 2 mm. The stimulation device can include at least one of a brushless motor, brushed motor, stepper motor, cam, linkage, piston, eccentric rotating mass (ERM) motor, linear resonant actuator (LRA), pump, or solenoid, to provide the mechanical stimulation.

In accordance with aspects of the instant disclosure, the stimulation device can deliver electrical stimulation. The electrical stimulation can be provided via contact with an electrode or a wired fabric disposed on the housing. The electrical stimulation can be selected from the group consisting of transcutaneous nerve stimulation (TENS), interferential therapy (IFT), pulsed electromagnetic field (PEMF), and Neuromuscular electrical stimulation (NMES). The electrical stimulation can be supplied with a frequency in a range of about 10 Hz to about 50 Hz. The electrical stimulation can have a duty cycle of about 1:3.5.

In some embodiments, the stimulation device can deliver thermal stimulation. The stimulation device can include a thermal stimulation device selected from the group consisting of a thermal resistive wire, an ultrasound generator, a shortwave diathermy (SWD), a microwave diathermy (MWD), a thermal pack, and a thermal compress.

In some embodiments, the sensor can detect a motion of or a force applied to the resistive exercise device or a predetermined parameter associated with the user. The sensor can be selected from the group consisting of goniometer, SpO2, pulse rate monitor, temperature sensor, accelerometer, gyroscope, magnetometer, pedometer, pressure sensor, bioimpedance, electrocardiography and electrodermal activity (EDA) sensor. The controller can have a user interface for receiving user inputs to control an operation of the stimulation device. The controller can be a remote controller for operating the stimulation device.

### BRIEF DESCRIPTION OF THE FIGURES

The accompanying drawings illustrate various embodiments of systems, methods, and embodiments of various other aspects of the disclosure. Any person with ordinary skills in the art will appreciate that the illustrated element boundaries (e.g., boxes, groups of boxes, or other shapes) in the figures represent one example of the boundaries. It may be that in some examples one element may be designed as multiple elements or that multiple elements may be designed as one element. In some examples, an element shown as an internal component of one element may be implemented as an external component in another, and vice versa. Furthermore, elements may not be drawn to scale. Non-limiting and non-exhaustive descriptions are described with reference to the following drawings. The components in the figures are not necessarily to scale, emphasis instead being placed upon illustrating principles.

FIG. 1 is a flow chart illustrating a method of use according to an embodiment;

FIG. 2 is a flow chart illustrating effects of stimuli in combination with exercise;

FIG. 3 is a schematic block diagram of an exercise device according to an embodiment;

FIG. 4A is a perspective view of an exercise device and stimulation device according to an embodiment;

FIG. 4B, FIG. 4C, and FIG. 4D are perspective views of an exercise device according to an embodiment;

FIG. 5A and FIG. 5B are perspective views of a stimulation device including mechanical stimulation;

FIG. 6A and FIG. 6B are perspective view of a stimulation device including thermal stimulation;

FIG. 7A and FIG. 7B are perspective view of a stimulation device including electrical stimulation;

FIG. 8A and FIG. 8B are perspective views of a stimulation device including any combination of mechanical, thermal, or electrical stimulation;

FIG. 9 is a perspective view of an alternative stimulation device;

FIG. 10A and FIG. 10B are perspective views of an exercise device and the stimulation device of FIG. 9, according to an embodiment;

FIG. 11A and FIG. 11B are perspective views of an exercise device and a stimulation device including mechanical stimulation;

FIG. 12A and FIG. 12B are perspective views of an exercise device and a stimulation device including thermal stimulation;

FIG. 13A and FIG. 13B are perspective views of an exercise device and a stimulation device including electrical stimulation; and

FIG. 14A and FIG. 14B are perspective views of an exercise device and a stimulation device including any combination of mechanical, thermal, or electrical stimulation.

### DETAILED DESCRIPTION

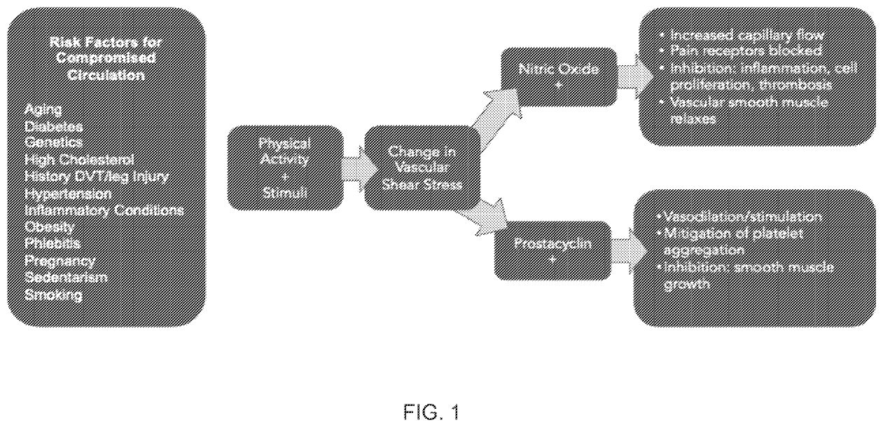

As shown in FIG. 1, the risk factors for compromised circulation can include aging, diabetes, genetics, high cholesterol, history DVT/leg injury, hypertension, inflammatory conditions, obesity, phlebitis, pregnancy, sedentarism, and smoking. Among the risk factors for compromised circulation, sedentarism is the one that is modifiable for a large portion of the general population. Therefore, the instant disclosure provides for the combination of physical activity and a stimuli, an antidote to sedentarism, may be reasonably prescribed as a therapeutic intervention for compromised circulation. Such a combination of physical activity and stimuli, as is described in full below, can lead to a change in vascular shear stress. For example, as shear stress changes, a complex biochemical cascade initiates, triggering the production of nitric oxide (NO), prostacyclin, and other molecules, all of which cause vascular smooth muscle relaxation. In addition, prostacyclin mitigates platelet aggregation. Causing the vascular smooth muscle to relax, NO decreases arteriole resistance, which increases capillary flow and blocks pain receptors.

Many studies have shed light on the potential for exercise to improve circulation, metabolic health, and immune function through action on the vascular endothelium. Physical activity, even in short bouts (i.e., 5 minutes), has been shown to prevent decline of vascular dilation function in sedentary individuals. Moreover, evidence suggests it has a significant positive impact on glycemic control and other metabolic indicators. Similarly, sustained periods (over 2 weeks) of any intensity physical activity is found to be sufficient to improve immune function. Except for long periods of high-intensity exercise, regular physical activity has been shown to decrease systemic inflammation, enhance healing, and improve response to viral infection.

Physical activity improves health by increasing circulation, stimulating biochemical cascades that promote cell function and quell inflammation. Muscle contractions and nerve activation increase vascular shear stress, facilitating venous return and inducing release of biomolecules with systemic effects: nitric oxide, endothelium derived hyperpolarizing factor, and prostacyclin. These biomolecules increase circulation of blood and lymphatic fluid facilitating exchange of critical nutrients, oxygen, waste, etc., while reducing inflammation, thrombus formation, edema, pain and endothelial dysfunction. These beneficial effects appear to be rooted in the modulation of vascular redox dynamics and inflammation associated with physical activity. As such, exercise is regarded as a means of addressing a wide array of chronic diseases, including neurodegenerative disease, age-related sarcopenia, and myriad other health conditions.

Accordingly, physical activity is repeatedly recommended in the literature as a viable therapeutic strategy. For instance, in the face of the recent COVID-19 outbreak, exercise is being recommended as a way of preparing for and countering cardiovascular and respiratory challenges common in the disease etiology. This approach is believed to be particularly valuable for older adults, who are already prone to sedentarism and immune challenges. Low to moderate resistance training is described in the literature as an effective strategy for maintaining muscle mass, strength, and functional capacity. It appear to be particularly valuable during care in a hospital or other clinical setting involving bedrest, where early intervention is associated with enhanced recovery. In short, prescribing physical activity represents an important opportunity to maintain independence and overall quality of life in both healthy and frail older adults, while slowing progression of disease and disability.

While physical activity appears to be a straightforward solution, the reality is that those most in need-often elderly or struggling with chronic health conditions already-may have difficulty exercising on a regularly. Thus, the instant disclosure provides for an important addition to post-operative and preventative care, to enhance overall recovery by combining physical activities with a stimuli for increased benefits to the patient. The instant device and methods can lead to shortened stays in acute care and reduce the need for a more compliant transition from hospital to home to prevent readmission. In some examples, a prescription of the instant device, even prior to a procedure, can support continuity of care because the device can be suitable for inpatient as well as out-patient use. Advantageously, the instant device can improve patient outcomes and patient satisfaction at a relatively low cost.

Such benefits can be achievable through the instant device, as such a device can be designed to increase circulation in a user-friendly, affordable package. For example, the instant device can be a simple device that can be designed to incentivize compliance so critical to success and bridge the need for supervision, which can be difficult to access for reasons such as lack of funds, lack of transportation, and infection risk.

In some embodiments, the instant device can be utilized in either a standing or sitting position. In an embodiment, the device can be intended for use in the sitting position. Further, in some embodiments, the instant device can be retrofitted with a stimuli pad for an added stimulation of the patient to the underlying resistance exercise. In some embodiments, the instant device can be used in the supine and prone position as well, to expand use to other settings and populations such as individuals on bedrest or in a healthcare facility.

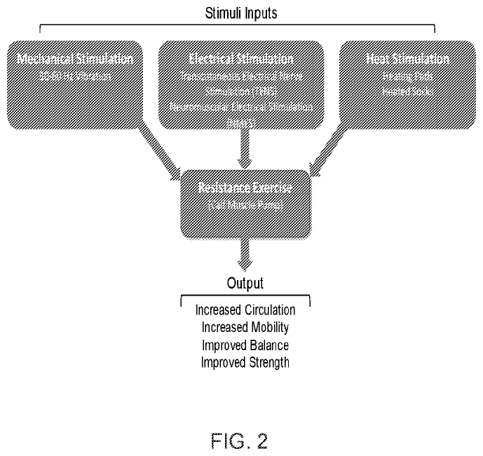

The device can drive vascular endothelial response and increase blood circulation in the lower periphery by combining mechanical stimulation, electrical stimulation, and thermos-stimulation with resistance exercise, as shown in FIG. 2. The combination of stimulation with resistance exercise, e.g., calf muscle pumps, can result in at least one of increased circulation, increased mobility, improved balance, or improved strength-as compared to one of the therapies alone.

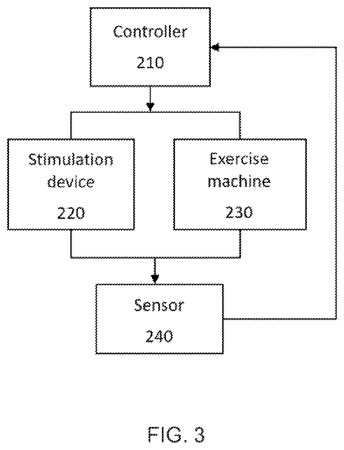

As shown in FIG. 3, in an embodiment, the instant device can include an integrated exercise system includes a controller 210, an exercise machine 230, a stimulation device 220 or stimulus member, and a sensor 240. Both the exercise machine 230 and the stimulation device 220 can be controlled by the controller 210 which takes in feedback information from the sensor 240 to make control decisions. In other embodiments, the exercise machine 230 may be a passive resistance exercise device which is not directly controlled by the controller 210. In an embodiment, the controller 210 may provide data to a telehealth monitor, a prompter, and a counter to provide instant feedback by way of the stimulation device. For example, as a user may need increased, or decreased, stimulation from the stimulation device as a function of detected motion from the sensor 240 on the exercise machine 230.

In other embodiments, the integrated exercise system may not employ a sensor 240, but can receive feedback and control from a user. For example, the user can independently control the stimulation device 220. Similarly, the integrated exercise system may not employ a separate controller. With or without the sensor and/or controller, the integrated exercise system can perform the following functionalities, alone or in various combinations:
Exercise+Thermal: Heat
Exercise+Thermal: Cold
Exercise+Electromagnetic: Electrical Stimulation
Exercise+Electromagnetic: Pulsed Electro-Magnetic Field
Exercise+Mechanical: Vibration
Exercise+Mechanical: Texture
Exercise+Mechanical: Acupressure Points

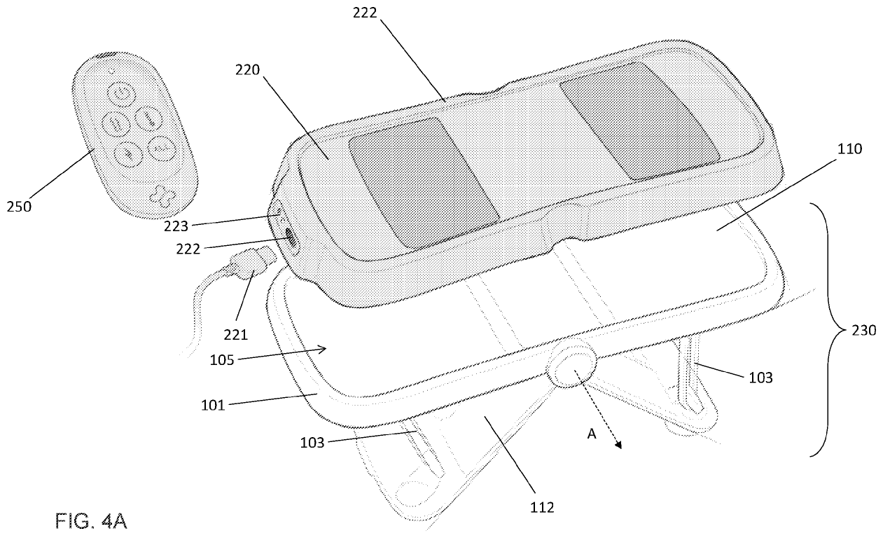

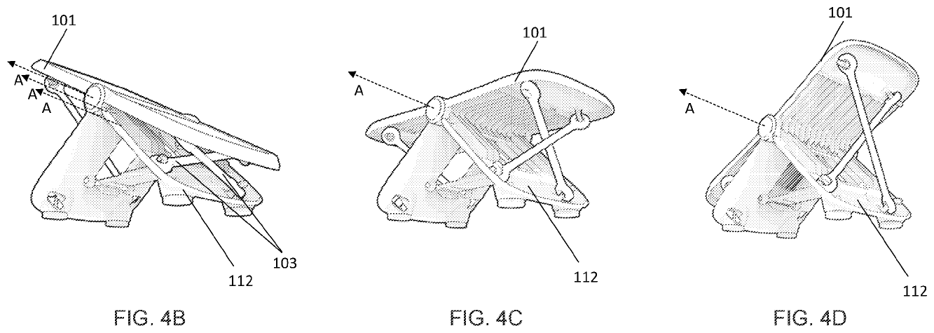

In an embodiment, the exercise machine 230 is a rotational pedal 101 as shown in FIG. 4A for exercising ankle, foot and/or leg of a user. Generally, the exercise machine 230 can be described as a resistive member that is in contact with a portion of a user's body and resists motion of the user's body. The pedal 101 can rotate around a pivot axis A at a top edge of a support member 110 approximate to a longitudinal center of the pedal 101. Four resistance mechanisms 103, e.g., resistance bands, can be connected to four corners of the pedal 101, respectively, to provide resistance to the pedal's rotation. Alternatively, the resistance mechanism 103 can include any of a stack of drag washers, springs, elastomeric bands, and a combination thereof. In an embodiment, the exercise machine 230 can be a portable exercise devices as shown in FIG. 4B, FIG. 4C, and FIG. 4D. The portable exercise device 230 shown in FIG. 4B, FIG. 4C and FIG. 4D illustrate the same device in three different configurations. The portable exercise device can be for exercising an ankle, a foot and/or a leg 10. Related methods can make use of at least one pedal 101 that is pivotably connected to a base 112 about a pivot axis A. The pedal 101 can have a neutral position relative to the pivot axis A and can be generally positioned such that the pivot axis is centrally located along a length of the pedal 101. When the pedal 101 is in the neutral position, as shown in FIG. 4C, the pedal 101 can be substantially parallel to the base 112 such that there is a space between the pedal 101 and the base. In this manner, the pedal 101 can be configured to rotate about the pivot axis A in a first direction away from the neutral position and toward the base 112 (where a first end of the pedal moves toward the base), as shown in FIG. 4B, and in a second direction away from the neutral direction and toward the base 112 (where a second end of the pedal moves toward the base), wherein the second direction is opposite the first direction, as shown in FIG. 4D. The pedal 101 may be used as a frame into which a stimulation device 220 is placed or integrated with. In some embodiments, the exercise device 230 can include a power source for powering various portions, including stimulators, sensors, and controls. The exercise device 230 may be powered by a variety of means including but not limited to secondary batteries (e.g., rechargeable batteries including lithium polymer and similar chemistries), primary batteries (e.g., alkaline AA batteries), wired AC to DC adapter (as a charging interface or a supplemental power source) or a solar power generator.

In some cases, a stimulation device 220 can be placed on top of the rotation pedal 101, as seen in FIG. 4A, such that it can be highly portable and useable with any number of different exercise machines 230. For example, exercise device 230 can be retrofitted to fit a stimulation device 220 within a pre-existing recess 105 on the rotation pedal 101. The stimulation device 220 can be locked to the rotation pedal 101 with any known mechanical or chemical fixation means. For example, one of the rotation pedal 101 or the stimulation device 220 can include clasps or detents that can be configured to interface with the other of the structure to retain the stimulation device 220 within the recess 105. While FIG. 4A illustrates the stimulation device 220 as being a separate device from the rotation pedal 101, it is contemplated that structure, and therefore the functionality, of the stimulation device 220 can be integrated directly into the rotation pedal 101. In some embodiments, the stimulation device 220 can be constructed from a compliant material housing 222 that provides a certain amount of give to provide comfort to a user. Portions of the exercise system 230, e.g., the stimulation device 220, that come in contact with user's body may be constructed of any of a variety of materials including but not limited to polyester, nylon, neoprene, sorbothane, poron, plastazzote, spenco, viscolas, lycra/spandex, foam, spacer mesh, antimicrobial mesh, and memory form. When fabric or form is used, superior durability, breathable, antimicrobial, comfort and water repellent may be preferred. The breathable, elastic, potentially antimicrobial materials can be surface cleaned and skin contact approved. In an embodiment, a top surface of the stimulation device 220 can be ergonomically formed, similar to the sole of a shoe to cradle and support a user's foot 10. In some embodiments, the housing 222 can contain all electrical and mechanical components of the stimulation device 220 described below.

The stimulation device 220 can provide any of mechanical, thermal, or electrical stimulation. In some cases, the stimulation device 220 can include an integrated battery (not shown) to provide power to the device. The integrated battery can be powered using a conventional power cord 221 and connection interface 222. Alternatively, the stimulation device 220 can be powered via a wired power line 221 plugged into an outlet. In some embodiments, the stimulation device 220 can include a remote-control device 250 for powering on respective stimuli or setting the specific therapy with any one stimuli. The remote control 250 can communicate with the stimulation device 220 using any known communication means including, but not limited to, infrared, BLUETOOTH, radio, etc. In other embodiments, the stimulation device 220 can include a user interface, e.g., buttons, directly on the device itself. In some embodiments, the stimulation device 220 can include a status indicator, e.g., status lights 223, to display to the user the current mode the device is operating in.

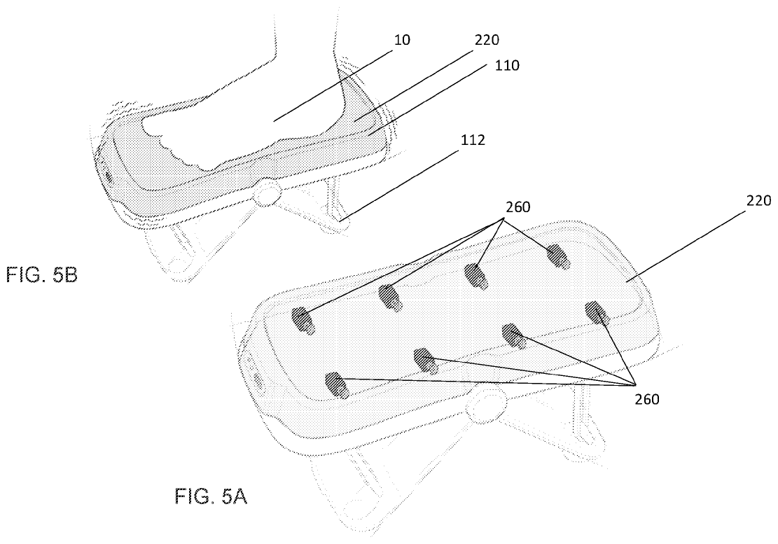

In some embodiments, as shown in FIG. 5A and FIG. 5B, the stimulation device 220 can controllably vibrate to provide a mechanical stimulation to a user's foot 10. In the above embodiments, in addition to vibration, mechanical stimulation can be provided in the forms of massaging, oscillation, percussion and pressure. In some embodiments, the stimulation device 220 can include one, or a combination of, eccentric rotating mass (ERM) motors, brushless motors, brushed motors, stepper motors, cams, linkages, pistons, linear resonant actuators (LRA), pumps and solenoids to generate motions for the mechanical stimulation. In some embodiments, the stimulation device can include between 4-10 mechanical actuators 260. In the illustrated embodiment, the stimulation device 220 can provide eight ERM motors, but any number of actuators can be included in the stimulation device 220. A top surface of the stimulation device can make contact with a user's body and may have groove patterns, spikes, textures, knobs and bumps to aid in the comfort of the user and transmission of the mechanical stimulation. The mechanical stimulation may also be augmented by shoe inserts such as gel insoles and liquid insoles. In some cases, the stimulation device 220 can provide a vibration frequency may vary in a range of about 10-60 Hz, in an embodiment, the range can be about 30-60 Hz; and a massage can be performed with about 25-50 pulses per second. In some embodiments, the mechanical stimulation device 220 can be synchronized with the pivotal motion between the foot support portion 110 and the base 112. Alternatively, the vibration frequency may be proportional to a plantar pressure measured at the foot support portion 110. For example, when a foot presses hard on the foot support portion, the amplitude of the vibration increases while the frequency of the vibration remain constant.

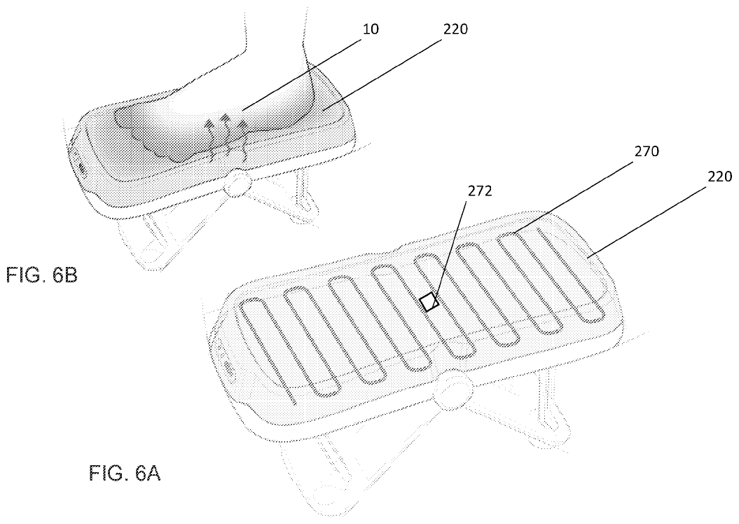

In some embodiments, as shown in FIG. 6A and FIG. 6B, the stimulation device 220 can provide for a thermal stimulation. Means of providing the thermal stimulation may include but is not limited to superficial heat, deep heat and cryotherapy. In the illustrated embodiment, the stimulation device 220 can include a single, or plurality, of resistive heating wires 270 arranged throughout the device 220. In some embodiments, the superficial heat may be introduced by chemical heat packs, microwaveable heat packs, hot water heat packs, electrical heat packs (flexible PTC, nichrome resistive, thick film and PCB trace heater), and insoles, footbeds, or socks with resistive heating elements 270. In some embodiments, the superficial heat may be controllable up to 140 degrees Fahrenheit. The deep heat may be introduced by ultrasound, shortwave diathermy (SWD), microwave diathermy (MWD), or electrical resistive wires 270 as is shown in FIG. 6A. The ultrasound frequency may have a range of 0.8-3 MHz and an upper limit of 3.0 W/cm² intensity. The shortwave diathermy (SWD) may have a frequency of 27.12 MHz. The microwave diathermy (MWD) may have a frequency in the range of 915-2456 MHz. The cryotherapy may be introduced by chemical cold packs, gel packs, clay packs, cold compresses and integrated chillers. The cold packs'temperature may be in the range of 40-60 degrees Fahrenheit. The cold compress may have a temperature of 45 degrees Fahrenheit and a pressure of up to 60 mmHg. In some embodiments, the stimulation device 220 can include a temperature sensor 272 that can measure the surface temperature to ensure that the stimulation device 220 does not heat or cool the device 220 above or below safe operating temperatures.

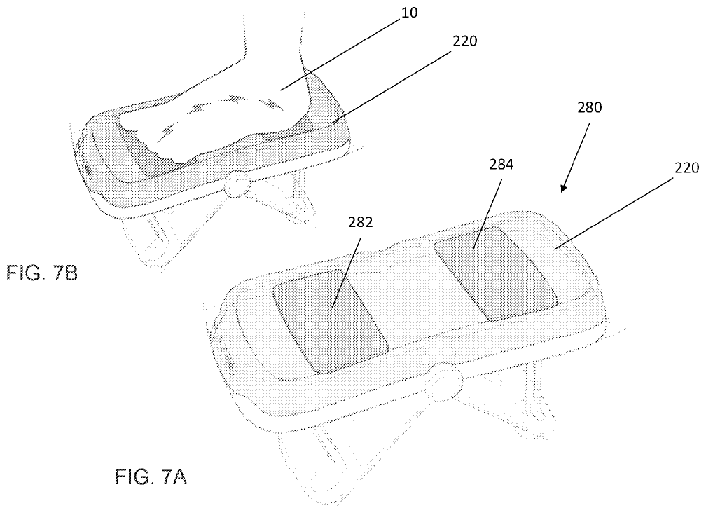

As shown in FIG. 7A and FIG. 7B, some embodiments of the stimulation device 220 can include electromagnetic, or electrical, stimulation components 280. In some embodiments, the stimulation device 220 can include at least two electrodes, a working electrode 282 and a ground electrode 284. In some embodiment, the stimulation device 220 can include at least two working electrodes 282. Alternatively, or additionally, means of providing the electromagnetic stimulation may include but are not limited to gel electrodes, wired fabrics, transcutaneous nerve stimulation (TENS), interferential therapy (IFT), neuromuscular electrical stimulation (NMES) and pulsed electromagnetic field (PEMF). The transcutaneous nerve stimulation (TENS) may use about 50-100 Hz frequency and approximately 50-300 μS pulse width. The interferential therapy (IFT) may use about 10-25 Hz frequency. The neuromuscular electrical stimulation (NMES) may use approximately 20-50 Hz frequency. The pulsed electromagnetic field (PEMF) may use a frequency from about 1 Hz to 10,000 Hz, and a maximum amplitude of about 1.19 μT and a maximum frequency of about 3.85 kHz.

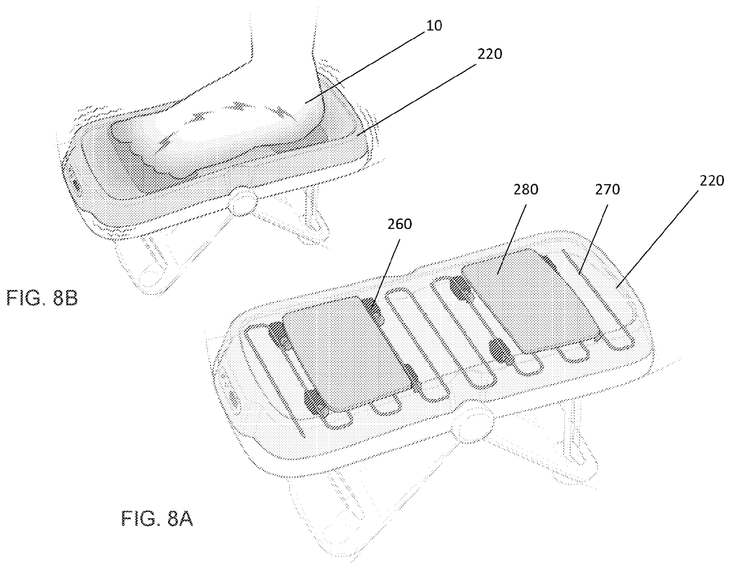

In some embodiments, as shown in FIG. 8A and FIG. 8B, the stimulation device 220 can include any combination of mechanical, thermal, or electrical stimulation. For example, the stimulation device 220 can provide at least two different stimulation types. As shown in FIG. 8A and FIG. 8B, the stimulation device 220 can include mechanical actuators 260, a resistive heating wire 270, and/or electrical stimulation components 280, along with all of their respective components. In such an embodiment, the stimulation device 220 can be controlled to provide each of the stimulations in a predefined sequence such that each stimulation is provided to the user individually, or the stimulation device 220 can provide any combination of stimulations simultaneously.

While not shown, in some embodiments, the stimulation device can include a pulse rate sensor and/or an oxygen sensor to measure physiological responses to the resistive exercises and the stimulation provided by the stimulation device 220. For example, the exercise system 230, and/or the stimulation device 220, may contain a variety of on-board sensors including but not limited to goniometer, SpO2, pulse rate monitor, temperature sensor, accelerometer, gyroscope, magnetometer, pedometer, pressure sensor (including strength measurement), bioimpedance, ECG and electrodermal activity (EDA). Data acquired by the on-board sensors can then be processed and transmitted to the controller for controlling the operations of the various stimulation devices, such as synchronizing the vibration with the pedal rotation speed, and maintain the thermal stimulation at a desired temperature. In some embodiments, an SpO2 can measure up to 100% O2; a pulse rate monitor can have an upper range of about 250 BPM; a temperature sensor can have a range up to about 122 degrees Fahrenheit; an accelerometer can measure accelerations up to approximately 100 g; a gyroscope can measure up to about 1,000 dps; a magnetometer can measure up to approximately 10 Gauss; a pressure sensor can measure up to approximately 1 psi; a bioimpedance sensor can measure up to approximately 250 kHz; an ECG can measure up to about 2 kHz; and an electrodermal activity (EDA) sensor can measure up to about 25 μS and about 3 Hz. Any combination of sensors may be used.

The sensor may record data for a physician to review or can be used to automatically, or actively, adjust any of the mechanical, thermal, or electrical stimulation based upon the sensed data. In some embodiments, the pedal 101 can be equipped with a motion sensor to detect movement thereof. The motion sensor can transmit the motion data to a controller which can control the mechanical, thermal, or electrical stimulation accordingly. In some examples, the stimulation device 220 can shift from one type of stimulation to another after a predetermined number of movements, or repetitions. In addition, the controller can include a timer which can turn off the stimulation device 220 after a predetermined number of repetitions, rest periods, or a fixed time period. In some embodiments, mechanical or electrical stimulation can be synchronized with the pedal's 101 rotation, i.e., when the pedal 101 rotates fast, the vibration frequency can increase, and vice versa. An amplitude of the vibration may be maintained as a constant while the frequency changes. Alternatively, the amplitude of the vibrations may be synchronized with the rotation of the pedal 101 while the frequency remains a constant. As an example, the vibration is in a drum mode.

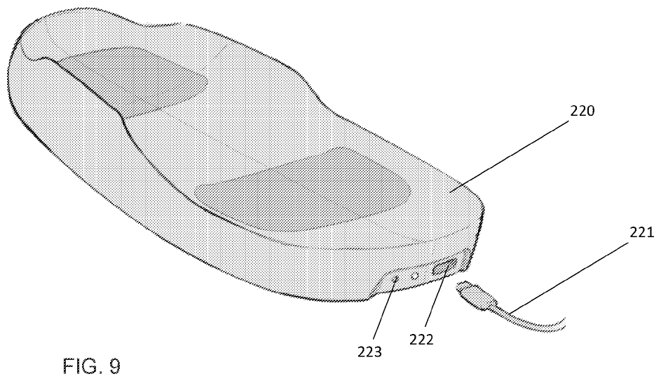

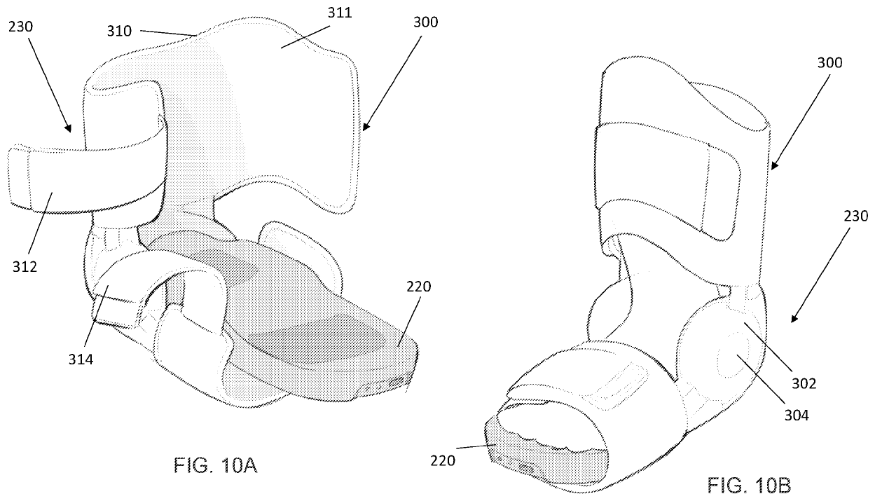

Turning to FIG. 9, FIG. 10A, and FIG. 10B, an alternative embodiment of an exercise device 230 and stimulation device 220 is shown. For the sake of brevity, a full description of the various aspects of the stimulation device 220 of FIG. 9, FIG. 10A, and FIG. 10B are not repeated.

In an embodiment, the exercise machine 230 can have an alternative foot support 300, as shown in FIG. 10A and FIG. 10B. The alternative exercise machine 230 can be beneficial to allow for the use to experience resistance when flexing their foot in either direction. As such, the device of FIG. 10A and FIG. 10B can be used in cases where the user is bedbound. In an embodiment, to protect the user's ankles, the respective hubs 302 (i.e., on each side of the device) and the hardware associated with the resistance mechanism 304 (i.e., that is seated within one of the hubs) can each be embedded within a soft compression molded padded liner 311 housed within the casing 310. As will be understood by those of ordinary skill in the art, the resistance mechanism 304 can be integrated within the device 230 using various additional materials and techniques. In further embodiments, to provide additional comfort and protection, the casing 310 may be formed about part of a compression molded liner 311 including a heel cup that is positioned between the leg cuff 312 and the foot cuff 314 connected to the cuffs via an elastic material. In this manner, in conjunction with the leg and foot cuffs 312, 314 the heel cup can be configured to provide a soft, enclosed, boot-like structure to hold the user's leg and foot securely in place. This outer structure, or casing, 300 may also be used to house a stimulation device 220. In some embodiments, stimulation device 220 can be inserted into an upper surface of the sole, formed as part of the casing 310. In some embodiments, the stimulation mechanisms of the stimulation device 220 can be integrated directly into the liner 311, which can be user replaceable. Alternatively, or additionally, portions of the stimulation device 220 can include, but are not limited to, textural features, vibrating elements, electrical stimulation electrodes, and thermal generators embedded into this casing and/or padding of the exercise device 230.

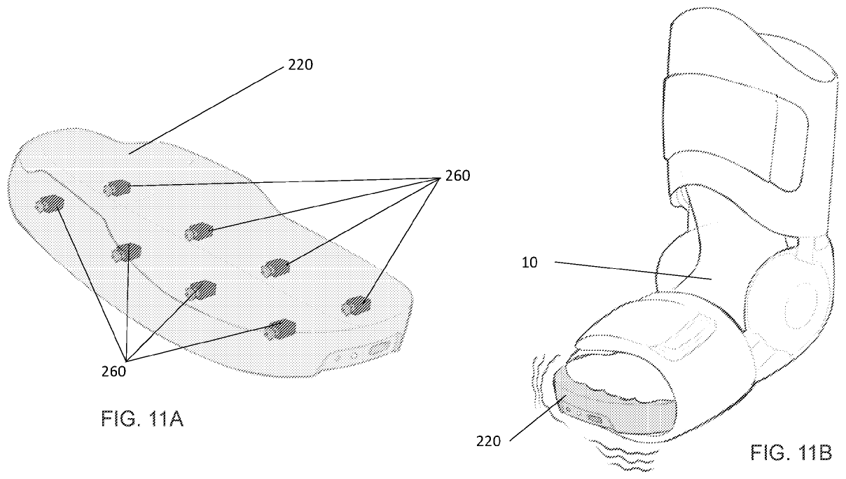

In some embodiments, as shown in FIG. 11A and FIG. 11B, the stimulation device 220 can include a mechanical stimulation actuator 260. In an embodiment, the stimulation device 220 can include a plurality of actuators 260, for example between 8-10 actuators. The mechanical actuators 260 can function to provide mechanical stimulation to the patient's leg and/or foot 10, as shown in FIG. 11B.

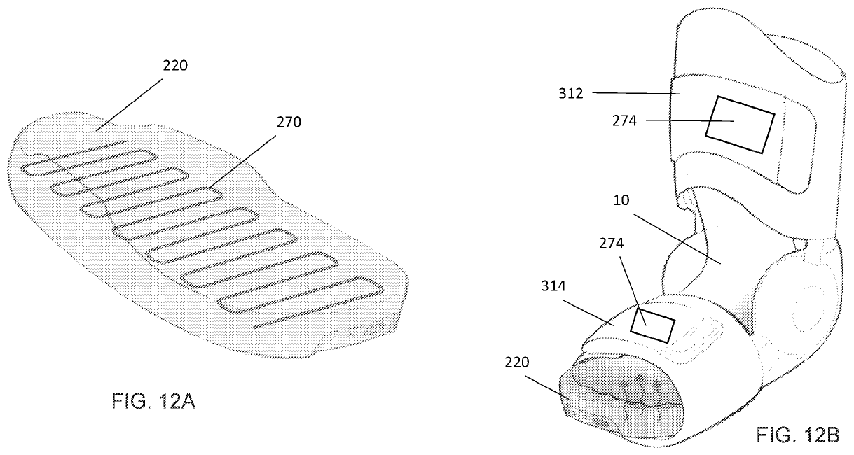

In other embodiments, as shown in FIG. 12A and FIG. 12B, the stimulation device 220 can provide thermal stimulation. In some embodiments, the stimulation device 220 can include at least one resistive wire 270. In an embodiment, the stimulation device 220 can include thermal stimulation elements 274 in at least one of the leg or foot cuffs 312, 314, to provide thermal stimulation to other portions of the user's appendage, e.g., the foot 10. The additional thermal stimulation elements 274 can be wired to, or wirelessly connected with, the stimulation device 220 or can be independent of the stimulation device 220.

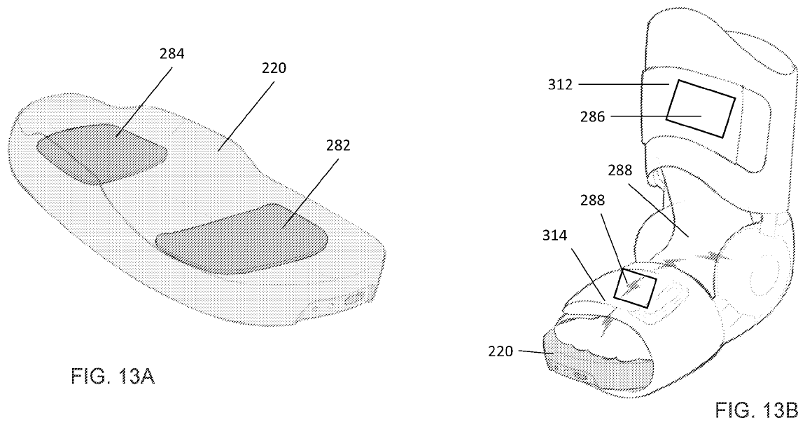

In some embodiments, as shown in FIG. 13A and FIG. 13B, the stimulation device 220 can provide electrical stimulation. In addition to the electrodes 282, 284 provided on the stimulation device 220 itself, the device can include electrodes 286, 288 in other areas of the casing 310, for example in at least one of the leg or foot cuffs 312, 314 to provide electrical stimulation to other areas of the leg or foot 10.

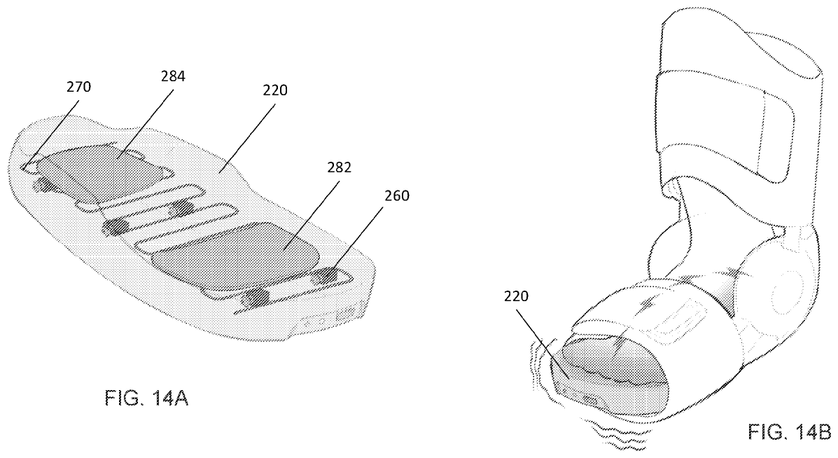

Similar to the embodiment of FIG. 8A and FIG. 8B, alternative foot support of FIG. 14A and FIG. 14B can include any combination of the mechanical stimulation 260, thermal stimulation 270, or electrical stimulation 282, 284 systems discussed above. While the illustrated embodiment shows all three types of stimulation systems integrated within the single stimulation device 220, it is contemplated that any combination of two stimulation systems can be combined. In some embodiments, the mechanical, thermal, and electrical stimulation systems can operate substantially the same as described above. In some embodiments, the stimulation device 220 can be powered and controls substantially the same as described herein.

It is contemplated that the stimulation devices discloses herein can be accommodated by most, if not all, exercise systems and are not intended to be limited to the foot. Moreover, while two foot exercise devices have been shown and discussed, it is contemplated that other exercise devices for the foot can be used. For example, the exercise machine 230 may accommodate a pair of foot pedals independently rotating around a pivot axis approximating to a bottom end of each foot pedal. Such exercise machine 230 can simultaneously exercise both lower limbs including ankles. Moreover, it is contemplated that the instant combination of a resistive, guided, exercise device 230 and a stimulation device 220 can be used for any joint of a user, e.g., hands, fingers, toes, knees, hips, wrists, elbows, shoulders, neck, etc.

As described with respect to FIG. 2, the instant device can boost circulation by combining, for example, foot-and-ankle resistance exercise with certain stimuli to the patient. Evidence supports use of these stimuli independently, and in combination with one another and resistance exercise, to boost circulation. For example, basic science and clinical work strongly suggests that low-level mechanical, thermal, or electrical stimulation of sensory neurons can significantly enhance local perfusion, muscle health, and assist in recovery of damaged nerve cells. There also is evidence that such stimuli can help to protect the neuromuscular system and potentially prevent stasis-induced deep vein thrombosis.

In some embodiments, resistance exercises in the lower periphery can enhance circulation by activating the Calf Muscle Pump (CMP). The CMP promotes venous return from the lower extremity and contributes to preload and cardiac output. Essentially, the CMP responds to the same hemodynamic shear stress that governs the health of all vascular endothelial tissue. Of the three natural muscle pumps of the lower limb, it is the most effective hemodynamically because of its high capacitance, the high pressures it can generate, and its positioning in the lower half of the limb, where the venous pressure is maximal. Function of the calf pump is affected by calf and foot muscle strength, ankle joint mobility and competency of the veins, nerve integrity, and ankle mobility. Ankle resistance exercises enhance circulation by activating the CMP, the primary means by which venous blood pumps back to the heart from the lower extremity (LE). It is highly hemodynamically effective. Strengthening the calf can lead to stronger contractions, greater blood flow, and a more efficient CMP function.

Muscular contractions and nerve activation increase vascular shear stress, facilitating venous return and inducing release of biomolecules with systemic effects: nitric oxide (NO), endothelium derived hyperpolarizing factor, and prostacyclin. These biomolecules increase blood flow and inhibit thrombosis, edema, pain and remove waste. All this can profoundly impact quality of life for a wide range of people.

Traditionally, the function of the calf muscle pump has been best achieved during heel-to-toe walking. Dorsiflexion of the foot before weight-bearing empties the distal calf pump; weight-bearing empties the foot pump; and plantar flexion following weight-bearing empties the proximal calf pump. The degree of flexibility of this joint is essential for the efficient functioning of the CMP. However, the combination of the aforementioned stimuli to the resistance exercises can lead to an improved outcome over resistance exercise alone, including heel-to-toe walking. Each of mechanical, thermal, and electrical stimulation can, on their own, provide certain benefits discussed below. Again, those benefits have been found to increase with the combination resistance exercise alone, or with one or more of the other stimuli.

For example, mechanical plantar stimulation may build upon baseline CMP activity, as it also enhances circulation, sensorimotor function, and muscle strength. In some embodiments, mechanical stimulation tested alone can activate mechanoreceptors, augmenting CMP activity. It can be shown that for sedentary individuals, low-frequency vibration, in particular, can have the effect of increasing peripheral blood flow and muscle blood volume associated with the CMP significantly in the calf, upper leg, and thoracic region. Such mechanical stimulation can generally be referred to as “micro-mechanical stimulation.” This mechanical energy can be provided at around 20-70 Hz with an amplitude of 10-100 microns in intermittent doses of 1-30 seconds with 1-30 second pauses. In some embodiments, the mechanical energy can be provided at around 45-50 Hz with an amplitude of 30-70 microns in intermittent doses of 10 seconds with 5-10 second pauses. When the mechanical stimulation device is positioned properly on the plantar surface of the foot (at a frontal postural reflex arc originating in the Meissner's corpuscles), this energy can stimulate nerve endings that activate the muscles of the CMP moving blood, lymphatic, and interstitial fluids.

Further to these circulatory benefits, other sensorimotor benefits of plantar vibration are shown that support individuals in becoming more ambulatory. For example, improvements can appear with respect to gait velocity and stride length, as well as step length, variability, asymmetry, and pitch contact. In general, mechanical stimulation can be a viable exercise alternative or adjunct for those who do not tolerate exercise well, such as neuromuscular patients.

In addition to mechanical stimulation, electrical stimulation(ES) can be an additional stimulation that may enhance circulation beyond that of baseline CMP activity. For example, ES has been found to engage muscle, relieve pain, and promote healthy circulation as well as neurological function. An important mechanism through which ES may achieve these benefits is through neuromuscular activation. In both nominally ambulatory individuals and those on bedrest, daily ES focused on muscle activation can have a significant positive impact on muscle mass preservation and perhaps strength. Through similar neuromuscular mechanisms, ES can be leveraged to restore motor function to areas of the body that have sustained nerve injury. As such, ES can aid in preventing a cycle of exercise intolerance increasing weakness, which in turn drives sedentarism and even more exercise intolerance, accelerating functional decline. For example, ES can be utilized therapeutically for muscle degeneration, either alone or in combination with dynamic mobilization or functional strength exercises, such as those resistance exercise described above. ES is seen as appealing for its low cost, efficiency and greater patient comfort than exercise alone.

Beyond these benefits, the muscle contraction induced by ES can improve circulation and oxygenation of tissue. Because ES can improve venous return, counteract venous stasis, and improve limb arterial inflow, it has been suggested as an effective method of thromboprophylaxis in the lower extremities. In certain applications, ES can result in a larger ejected blood volumes from the veins and soleal sinuses (a common site of thrombosis) than does intermittent pneumatic compression alone, with greater patient comfort and user friendliness.

Much like electrical and mechanical stimulation, application of heat or cooling to the lower extremity has been shown to promote healthy circulation, muscle health, and analgesic effects. For example, thermotherapy interventions can contribute to muscle recovery (e.g., by attenuating cellular damage and protein degradation) while enhancing capillarization, muscle mass, and mitochondrial function in skeletal muscle models. Thermotherapy can also reduce lipid and glycemic markers of disease. In addition, thermotherapy can be appropriate for a broad range of the healthy population as well as for those recovering from health conditions, it is seen as a cost effective, accessible, and safe therapeutic option for a variety of indications. For example, thermotherapy can be a viable alternative to exercise for those who are exercise intolerant. In addition, or alternatively, thermotherapy can be beneficial in combination with exercise improving key indicators including walking distance, overall functional ability, and resting blood pressure.

These favorable effects of thermotherapy, or thermal therapy, can be attributed to action on the vascular endothelium and hence, the CMP. Passive heating increases limb blood flow; the resulting change in shear rate induces vessel dilation via both sensory neural and NO pathways. In some embodiments, the thermotherapy can be heat therapy which can be used to help find the delicate balance between the physical and biochemical cascades in the vascular endothelium that govern vessel dilation and constriction.

Each of mechanical, electrical, and heat stimulation of the lower extremity can be used as stand-alone treatments or simply in combination with exercise, but each of those stimulations can also be used in combination with one another. Combination of these stimuli with exercise can improve blood flow concomitantly with key clinical endpoints: pain, walking distance, overall functional ability, and resting blood pressure.

The aforementioned stimuli have been found to interact synergistically with one another to maximize vascular function. Increases in blood flow are often associated with clinical improvement. For example, in the case of wound healing, electrical stimulation was found to be more effective when used in a warm room, as evidenced by greater blood flow both during and after application of electrical stimulation, relative to its use in colder rooms.

Just as a synergistic effect was found between heat and electrical stimulation, the combination of thermotherapy and vibration can additionally be beneficial. Heat and vibration in combination are associated with substantially elevated skin and muscular blood flow in both young and older adults. For example, an elevated level of skin and muscular blood flow can be shown with both during and several minutes after treatment including both thermotherapy and vibration. Furthermore, thermotherapy in combination with vibration can enhance plantar sensitivity.

In an example, use of the instant exercise device 230 can demonstrate a meaningful boost in lower extremity venous return. For example, twelve healthy adults were evaluated using the device to determine the effect on maximum blood flow velocity and vessel diameter of the right popliteal vein. Results with low level resistance and a rate of 30 cycles per minute for 1 minute yielded an average increase in blood flow of approximately 150% above resting. Additional anecdotal tests demonstrated a 410% increase in venous return at a higher resistance setting and a 401% increase by doubling the rate of motion.

Combining resistance exercise with select passive stimuli can improve blood flow more than resistance exercise alone, bringing significant health benefits. For example, exercise combined with adjunctive electrical stimulation (eStim) can roughly triple blood flow and yielded 2.5 times pain score reduction, relative to exercise alone. Specifically, exercise without adjunctive eStim was associated with a 15% short-term improvement in blood flow and 6% reduction in diabetic neuropathy pain score. In contrast, exercise combined with eStim resulted in 54% blood flow increase with 51% reduction in pain score. The disclosed device will be used to further explore the therapeutic value of such combinatorial approaches to enhance circulation.

The instant disclosure can be used to assess the resistance exercise device 230 that can offer integrated heat, vibration, and/or electrical stimulation to boost circulation. Circulatory effects can be assessed using Doppler ultrasound evaluation of blood flow taken before and after exercise.

### Mechanical Stimulation

As mentioned above, mechanical stimulation in the form of low frequency, low amplitude vibrations can have a significant positive impact on peripheral blood flow. This positive impact can be achieved with waves that have an amplitude of 2 mm and frequency of 50 Hz administered for 5 minutes total with a duty cycle that alternates between 10 seconds or less on and a pause that is roughly the same length or less.

In one example, a duty cycle can be 600 ms on and 600 ms off, which although different from target values outlined above, suggest that it, too has a positive effect on peripheral blood flow. These improvements were seen at both 10 Hz and 40 Hz. In another example, elderly individuals performing exercises on whole body vibration platforms experienced increases in muscular strength (38%) and cardiorespiratory fitness as well as preservation of muscular power, comparable to or exceeding exercise alone.

### Electrical Stimulation

Per the prior discussion, electrical stimulation applied superficially on the skin (transcutaneously) also has been shown to enhance peripheral blood flow. Significant increases in perfusion have been found at frequencies as low as 4 Hz. in some examples, effective frequencies can be in the range of 10 Hz to 50 Hz; for example, 35 Hz. In some embodiments, the electrical stimulation can be provided at a level that would just engage the motor threshold, eliciting a slight visible twitch, which was comfortably tolerated by participants, alternatively, the system can be used to provide twice the motor threshold.

Other variables can include a range of pulse width/phase duration of 200 ms to 1,000 ms with peak blood flow at 300 ms. Duty cycles can be, as well, but generally can have a ratio of “on” time to “off” time of 1:3.5. For example, utilizing a pulse width of 300 ms with a biphasic symmetric square waveform with ramp up and ramp down times of 2 s each, with a duty cycle of 12 s on and 48 s off.

In one embodiment, the system can utilize the QUATTRO 2.5 Electrotherapy device DQ8450 (Roscoe Medical). Applying single use gel electrodes to the plantar surface of the foot. In one embodiment, only the frequency can be varied as follows: 10 Hz and 50 Hz. For example, a combination of eStim with exercise was found to lead to 40% increase in walking distance among those with PAD. It also is associated with 50% reduction in pain score among those with DPN, markedly higher than the 6% increase seen with exercise alone.

### Thermal Stimulation

Perhaps the most straightforward is the range of values for the parameter of heat. Heat has been evaluated for a formal role in medical treatment and management of chronic health conditions. In an example, elderly individuals with PAD randomized to 12 weeks of spa bathing or supervised exercise exhibited increases in total and pain-free walking distance increased of >40 m with both interventions. Flow mediated vessel dilation increased 17% and 55% respectively, as did vascular endothelial growth factor (46% and 243%). Combining heat with exercise enhanced outcomes. It has been found that a low level, gradual application of heat that raises body to temperature to around 102° F. is safest and most effective. This is often achieved by applying localized heat with a pad or bath set to a temperature of 104° F. to 119° F. Sauna therapy in the range of 140° F. has been found effective, too. The range of heat settings for many indoor heated slippers is between 94° F. and 120° F. In general, those with diminished sensation, are advised to use lower settings with any heat source to avoid injury. In this example, heat can be applied by the stimulation device 220. For the purposes of this example, heat can be applied at two different levels: 93° F. to 100° F. and 113° F. to 135° F. While these examples of thermal stimulation relate to the application of heat, there are additional and different benefits to the application of cold therapy as well.

As utilized herein, the terms “comprises” and “comprising” are intended to be construed as being inclusive, not exclusive. As utilized herein, the terms “exemplary”, “example”, and “illustrative”, are intended to mean “serving as an example, instance, or illustration” and should not be construed as indicating, or not indicating, a preferred or advantageous configuration relative to other configurations. As utilized herein, the terms “about”, “generally”, and “approximately” are intended to cover variations that may existing in the upper and lower limits of the ranges of subjective or objective values, such as variations in properties, parameters, sizes, and dimensions. In one non-limiting example, the terms “about”, “generally”, and “approximately” mean at, or plus 10 percent or less, or minus 10 percent or less. In one non-limiting example, the terms “about”, “generally”, and “approximately” mean sufficiently close to be deemed by one of skill in the art in the relevant field to be included. As utilized herein, the term “substantially” refers to the complete or nearly complete extend or degree of an action, characteristic, property, state, structure, item, or result, as would be appreciated by one of skill in the art. For example, an object that is “substantially” circular would mean that the object is either completely a circle to mathematically determinable limits, or nearly a circle as would be recognized or understood by one of skill in the art. The exact allowable degree of deviation from absolute completeness may in some instances depend on the specific context. However, in general, the nearness of completion will be so as to have the same overall result as if absolute and total completion were achieved or obtained. The use of “substantially” is equally applicable when utilized in a negative connotation to refer to the complete or near complete lack of an action, characteristic, property, state, structure, item, or result, as would be appreciated by one of skill in the art.

Numerous modifications and alternative embodiments of the present disclosure will be apparent to those skilled in the art in view of the foregoing description. Accordingly, this description is to be construed as illustrative only and is for the purpose of teaching those skilled in the art the best mode for carrying out the present disclosure. Details of the structure may vary substantially without departing from the spirit of the present disclosure, and exclusive use of all modifications that come within the scope of the appended claims is reserved. Within this specification embodiments have been described in a way which enables a clear and concise specification to be written, but it is intended and will be appreciated that embodiments may be variously combined or separated without parting from the invention. It is intended that the present disclosure be limited only to the extent required by the appended claims and the applicable rules of law.

What is claimed is:

1. An exercise apparatus comprising:
a resistive member resisting a motion of a part of a user's body; and
a stimulation device removably engaging both the resistive member and the part of the user's body for providing at least two different stimulations to the user's body, where the at least two different stimulations are different than one another.

2. The exercise apparatus of claim 1, wherein one of the at least two different stimulations is mechanical stimulation in the form of a vibration frequency range of about 30-60 Hz.

3. The exercise apparatus of claim 2, wherein the mechanical stimulation has a duration of approximately 5 minutes.

4. The exercise apparatus of claim 2, wherein the mechanical stimulation has an amplitude of approximately 2 mm.

5. The exercise apparatus of claim 2, wherein the stimulation device includes at least one of a brushless motor, brushed motor, stepper motor, cam, linkage, piston, eccentric rotating mass (ERM) motor, linear resonant actuator (LRA), pump, or solenoid, to provide the mechanical stimulation.

6. The exercise apparatus of claim 2, wherein the mechanical stimulation is actuated as function of a motion of the resistive member.

7. The exercise apparatus of claim 1, wherein one of the at least two different stimulations is electrical stimulation.

8. The exercise apparatus of claim 7, wherein the electrical stimulation is provided via contact with an electrode or a wired fabric disposed on the stimulation device.

9. The exercise apparatus of claim 7, wherein the electrical stimulation is selected from the group consisting of transcutaneous nerve stimulation (TENS), interferential therapy (IFT), pulsed electromagnetic field (PEMF), and Neuromuscular electrical stimulation (NMES).

10. The exercise apparatus of claim 7, wherein the electrical stimulation is supplied with a frequency in a range of about 10 Hz to about 50 Hz.

11. The exercise apparatus of claim 10, wherein the electrical stimulation has a duty cycle of about 1:3.5.

12. The exercise apparatus of claim 7, wherein the electrical stimulation is provided as function of a motion of the resistive member.

13. The exercise apparatus of claim 1, wherein at least one of the at least two different stimulations is thermal stimulation.

14. The exercise apparatus of claim 13, wherein the stimulation device includes a thermal stimulation device selected from the group consisting of a thermal resistive wire, an ultrasound generator, a shortwave diathermy (SWD), a microwave diathermy (MWD), a thermal pack, and a thermal compress.

15. The exercise apparatus of claim 13, wherein the thermal stimulation is provided as function of a motion of the resistive member.

16. The exercise apparatus of claim 1 further comprising:
a sensor configured to detect a predetermined parameter associated with the user's body; and
a controller configured to receive detected data from the sensor and control an operation of stimulation according to at least a part of the detected data.

17. The exercise apparatus of claim 16, wherein the sensor detects a motion of or a force applied to the resistive member or a predetermined parameter associated with the user.

18. The exercise apparatus of claim 16, wherein the sensor is selected from the group consisting of goniometer, SpO2, pulse rate monitor, temperature sensor, accelerometer, gyroscope, magnetometer, pedometer, pressure sensor, bioimpedance, electrocardiography and electrodermal activity (EDA) sensor.

19. The exercise apparatus of claim 16, wherein the controller has a user interface for receiving user inputs to control an operation of the stimulation device.

20. The exercise apparatus of claim 19, wherein the controller is a remote controller for operating the stimulation device.

21. The exercise apparatus of claim 1, wherein the at least two different stimulations are a predefined program that is associated with an exercise and the user.

22. An exercise apparatus comprising:
a pivotable foot rest;
a resistive member coupled to and resisting a motion of the pivotable foot rest;
a stimulus member engaging with the pivotable foot rest, the stimulus member being configured to:
provide a mechanical stimulation to a user's body;
provide an electrical stimulation to the user's body; and
provide a thermal stimulation to the user's body;
a sensor configured to detect a predetermined parameter associated with the user's body; and
a controller configured to receive detected data from the sensor and control an operation of the stimulus member according to at least a part of the detected data.

23. The exercise apparatus of claim 22, wherein the pivotable foot rest further includes
a casing for partially surrounding the user's body;
a liner inserted within the casing for receiving the user's body; and
at least one cuff for securing the casing to the user's body.

24. The exercise apparatus of claim 23, wherein the sensor is disposed in one of the at least one cuff or the liner.

25. The exercise apparatus of claim 23, further including a second stimulus member disposed within at least one of the at least one cuff or the liner.

26. The exercise apparatus of claim 22, wherein the stimulus member is one of removably engaging top surface of the pivotable foot rest or integrated into the pivotable foot rest.

27. A method comprising:
providing a pivotable pedal having a resistance member for resisting pivoting of the pivotable pedal;
pivoting a part of a user's body to pivot the pivotable pedal; and
generating at least two different stimulations to the part of the user's body with a stimulation device.

28. The method of claim 27, wherein the at least two different stimulations include two of mechanical stimulation, thermal stimulation, and electrical stimulation.

29. The method of claim 27,
detecting a predetermined parameter of the user with a sensor associated with the stimulation device; and
controlling the at least two different stimulations as a function of the detected predetermined parameter.

30. A stimulus member, the stimulus member comprising,
a housing configured to removably engage a resistive exercise device;
a stimulation device configured to deliver at least one of a mechanical stimulation, electrical stimulation, or thermal stimulation to a user's body;
a sensor configured to detect a predetermined parameter associated with the user's body; and
a controller configured to receive detected data from the sensor and control an operation of the stimulation device according to at least a part of the detected data.

31. The stimulus member of claim 30, wherein the stimulation device delivers mechanical stimulation in the form of a vibration frequency range of about 30-60 Hz.

32. The stimulus member of claim 31, wherein the mechanical stimulation has a duration of approximately 5 minutes.

33. The stimulus member of claim 31, wherein the mechanical stimulation has an amplitude of approximately 2 mm.

34. The stimulus member of claim 31, wherein the stimulation device includes at least one of a brushless motor, brushed motor, stepper motor, cam, linkage, piston, eccentric rotating mass (ERM) motor, linear resonant actuator (LRA), pump, or solenoid, to provide the mechanical stimulation.

35. The stimulus member of claim 30, wherein the stimulation device delivers electrical stimulation.

36. The stimulus member of claim 35, wherein the electrical stimulation is provided via contact with an electrode or a wired fabric disposed on the housing.

37. The stimulus member of claim 35, wherein the electrical stimulation is selected from the group consisting of transcutaneous nerve stimulation (TENS), interferential therapy (IFT), pulsed electromagnetic field (PEMF), and Neuromuscular electrical stimulation (NMES).

38. The stimulus member of claim 35, wherein the electrical stimulation is supplied with a frequency in a range of about 10 Hz to about 50 Hz.

39. The stimulus member of claim 38, wherein the electrical stimulation has a duty cycle of about 1:3.5.

40. The stimulus member of claim 30, wherein the stimulation device delivers thermal stimulation.

41. The stimulus member of claim 40, wherein the stimulation device includes a thermal stimulation device selected from the group consisting of a thermal resistive wire, an ultrasound generator, a shortwave diathermy (SWD), a microwave diathermy (MWD), a thermal pack, and a thermal compress.

42. The stimulus member of claim 30, wherein the sensor detects a motion of or a force applied to the resistive exercise device or a predetermined parameter associated with the user.

43. The stimulus member of claim 30, wherein the sensor is selected from the group consisting of goniometer, SpO2, pulse rate monitor, temperature sensor, accelerometer, gyroscope, magnetometer, pedometer, pressure sensor, bioimpedance, electrocardiography and electrodermal activity (EDA) sensor.

44. The stimulus member of claim 30, wherein the controller has a user interface for receiving user inputs to control an operation of the stimulation device.

45. The stimulus member of claim 30, wherein the controller is a remote controller for operating the stimulation device.
